(19) United States
(12) Patent Application Publication
(10) Pub. No.: US 2023/0255870 A1
(43) Pub. Date: Aug. 17, 2023

(54) TEMPORARY ANTI-BRASSY AND RESTORATIVE BLUE OIL FOR BLACK OR DARK HAIR WITH HIGHLIGHTS OR BALAYAGE

(71) Applicant: Lilliana Rehder, Tucson, AZ (US)

(72) Inventor: Lilliana Rehder, Tucson, AZ (US)

(21) Appl. No.: 18/060,301

(22) Filed: Nov. 30, 2022

Related U.S. Application Data
(60) Provisional application No. 63/309,547, filed on Feb. 12, 2022.

(51) Int. Cl. A61K 8/46; A61Q 5/12; A61Q 5/00; A61K 8/92

(52) U.S. Cl. CPC A61K 8/463; A61Q 5/12; A61Q 5/004; A61K 8/922; A61K 2800/43; A61K 2800/882

(57) ABSTRACT

Various solutions have been proposed for cancellation of brassy tones in hair strands. Formulations disclosed herein are natural alternatives to potentially damaging anti-brassy hair products. A temporary anti-brassy and restorative blue oil may hydrate and improve the texture of damaged, dry hair caused by bleaching or lightening processes. The blue pigments in this composition are configured to enhance an original salon color until the next salon visit.

### CROSS-REFERENCES TO RELATED APPLICATIONS

This application is a non-provisional and claims benefit of U.S. Provisional Application No. 63/309,547 filed Feb. 12, 2022 the specification of which is incorporated herein in their entirety by reference.

### FIELD OF THE INVENTION

The present invention relates to temporary anti-brassy and restorative hair oils. More specifically, the present invention relates to blue temporary anti-brassy and restorative hair oils.

### BACKGROUND OF THE INVENTION

Hair coloring techniques such as balayage, ombre, and highlights have become popular. However, frequent application of these sorts of coloring techniques may damage the hair, leaving it dry and frizzy. Many products on the market claim to avoid color fade after washes, but they often also dry the hair. For that reason, it may be recommended to limit use of such products to once a week.

People with dark or black hair tend to have red pigments in their hair. When black hair is color treated or bleached, it tends to return to those red/orange brassy tones after about 2 weeks. Compositions of the present invention cancel out the brassiness, leaving the hair restored and shiny from the moment of application, resulting in brighter color, and glossier, more beautiful hair.

There are various products on the market designed to cancel out brassy hair, most of them in the form of purple shampoos. However, purple pigments work better with blonde hair to cancel out yellow tones, and blue pigments work better for black or dark hair to cancel undesirable brassy tones. There are very few products in the market for this purpose, and most are in the form of shampoos. There is a need for new formulations which are not shampoos, conditioners, or hair masks, but instead something a user can apply and leave in for a glossier effect, and that can be used as often as desired.

### BRIEF SUMMARY OF THE INVENTION

It is an objective of the present invention to provide compositions and methods that allow for hair strengthening and color enhancement, as specified in the independent claims. Embodiments of the invention are given in the dependent claims. Embodiments of the present invention can be freely combined with each other if they are not mutually exclusive.

In some embodiments, the present invention features an anti-brassy color enhancer and restorative oil for black or dark hair with balayage, ombre, or highlights. Embodiments may include natural options, such as a formulation using 100% organic and certified cosmetic oils mixed with dextrose and FD&C Blue 1 Lake. This composition is designed to not only nourish the hair, but also to hydrate it and repair the damage caused by ammonia and other chemicals when lightening or bleaching the hair. The composition may, in some embodiments, be used as often as desired. The formulation may be designed specifically for black or dark hair since the brassy tones in hair of these colors are different from brassy tones in blonde hair.

One of the unique and inventive technical features of the present invention is the mixing of a blue coloring agent with a cosmetic oil base. Without wishing to limit the invention to any theory or mechanism, it is believed that the technical feature of the present invention advantageously provides for restoration of hair glossiness while simultaneously providing a temporary anti-brassy effect. None of the presently known prior references or work has the unique inventive technical feature of the present invention.

Mineral cosmetic oils may have an essential role in stopping hair from breaking. While the scalp naturally produces oil, sometimes an uneven balance of oils or environmental factors can cause a person's hair to be dry, frizzy, or brittle. Additionally, using some types of mineral cosmetic oils on hair can coat the hair follicles to prevent damage from UV rays, and prevent hair from breaking as it grows out. Some oils are better than others. For example, saturated and monosaturated oils are generally better able to penetrate the hair than polyunsaturated fats are. In some embodiments, compositions of the present invention may contain natural ingredients, no sulfates, no parabens, and be cruelty-free, alcohol-free, silicon-free, keratin-free, peroxide-free, and ammonia-free.

Furthermore, the prior references teach away from the present invention. For example, there is a potential concern that applying an oil formulation with concentrated blue pigments might stain clothes, pillowcases, blankets, hats, etc. However, once the formulations of the present invention are applied and absorbed by the hair, they do not leave any residue and will not stain. Additionally, some may believe that the use of cosmetic oil formulations may cause greasy or heavy hair. However, when formulations of the present invention are applied as directed, they may avoid causing greasy or heavy hair because the oils used in the composition are very light and specifically selected oils.

Furthermore, the inventive technical feature of the present invention contributed to a surprising result. For example, while application of formulations of the present invention to damp hair causes an immediate difference in hair color, it may also make the hair more manageable, help to reduce tangles, decrease frizz, lock moisture in the hair, or any combination thereof. Additionally, the formulations of the present invention may prevent hair from breaking by acting as a lubricant. Furthermore, use of the formulations of the present invention may extend the amount of time between salon visits by extending the amount of time that a salon treatment maintains a suitable look.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### DETAILED DESCRIPTION OF THE INVENTION

In one embodiment, the present invention features an oil-based hair treatment composition. As a non-limiting example, the composition may include greater than 90 weight percent of a cosmetic oil base; and less than 10 weight percent of a coloring agent. In various embodiments, the composition may include greater than about 75, 76, 77, 78, 79, 80, 81, 82, 83, 84, 85, 86, 87, 88, 89, 90, 91, 92, 93, 94, 95, 96, 97, 98, or 99 weight percent of a cosmetic oil base. In various embodiments, the composition may include less than 25, 24, 23, 22, 21, 20, 19, 18, 17, 16, 15, 14, 13, 12, 11, 10, 9, 8, 7, 6, 5, 4, 3, 2, or 1 weight percent of a coloring agent. In preferred embodiments, the composition may provide strengthening and color enhancement of hair.

In additional embodiments, the coloring agent may be dissolved or suspended within the oil base. In some embodiments, the coloring agent may include a blue food coloring powder or other colored powder. As a non-limiting example, the coloring powder may include about 61 weight percent dextrose and about 39 weight percent FD&C Blue 1 Lake.

In some embodiments, the cosmetic oil base may include one or more pure and certified organic cosmetic oils. As a non-limiting example, the cosmetic oil base may include avocado oil, argan oil, jojoba oil, almond oil, grapeseed oil, or a combination thereof. Additionally, the composition may comprise less than 5 weight percent essential oil. As a non-limiting example, the composition may include about 11 drops of lavender oil and about 11 drops of vanilla oil per 50 grams.

In one embodiment, about 50 grams of the composition may include about 10-20 mL of avocado oil; about 10-20 mL of argan oil; about 5-10 mL of jojoba oil; about 5-10 mL of almond oil; about 5-10 mL of grapeseed oil; and about 1-6 grams of a blue food coloring agent.

In selected embodiments, the present invention may feature a method of hair treatment. As a non-limiting example, the method may include: identifying hair in need of treatment; providing an oil-based hair treatment composition; and applying about 3 to 10 drops of the composition to hair and distributing the composition throughout the hair. As used herein, the term “hair in need of treatment” refers to hair which displays signs of damage, dehydration, or dryness, signs that brassy tones have or will appear, or a combination thereof.

The oil-based hair treatment composition may include greater than 90 weight percent of a cosmetic oil base; and less than 10 weight percent of a coloring agent. As a non-limiting example, the coloring agent may comprise a blue coloring agent. In preferred embodiments, the hair may be damp prior to application of the composition (e.g., a preliminary step may include moistening the hair). In alternative embodiments the composition may be applied to dry hair. Disposable gloves may be worn during application of the composition. The method may include washing hands with water and soap for about 20 seconds after application of the composition.

The present invention may feature a method of preparing an oil-based hair treatment composition. As a non-limiting example, the method may include: providing one or more cosmetic oils; providing one or more coloring agents; and mixing the one or more cosmetic oils with the one or more coloring agents to provide an oil-based hair treatment composition. In one embodiment, the one or more cosmetic oils may include avocado oil, argan oil, jojoba oil, almond oil, grapeseed oil, or a combination thereof. In selected embodiments, the one or more cosmetic oils may comprise greater than 90 weight percent of the composition and the one or more coloring agents may comprise less than 10 weight percent of the composition. In some preferred embodiments, the composition is not a shampoo and lacks surfactants or other typical shampoo components.

As a non-limiting example, 50 grams of the composition may include: about 5-25 mL of avocado oil; about 5-25 mL of argan oil; about 5-25 mL of jojoba oil; about 5-25 mL of almond oil; about 5-25 mL of grapeseed oil; and about 0.5-10 grams of a blue food coloring agent. In some embodiments, the method may additionally include providing one or more essential oils and mixing the one or more essential oils with the one or more cosmetic oils with the one or more coloring agents.

### EXAMPLE

The following is a non-limiting example of the present invention. It is to be understood that said example is not intended to limit the present invention in any way. Equivalents or substitutes are within the scope of the present invention.

The hair composition of this example is for black or dark hair that has been professionally lightened or bleached at the salon. The science behind this example is that after hair coloring, the darker the original hair color, the more of a challenge it is to lift the underlying pigment out of the hair, making darker color hair more prone to brassy color.

When hair color starts to fade, it can turn brassy, and natural hair pigments such as red tones can appear. Blue pigment cancels out orange or red tones in hair and ensures that the cool and lighter tones are kept. This works because colors that are opposite each other in the color wheel cancel each other out, so that blue cancels out red and orange, just as purple cancels out yellow or green. Sun exposure, pool chlorine, shower water, ocean water, pollution, and the use of electric irons and curlers may also contribute to brassy hair.

The intention of this example product is to help people who have black or dark hair to maintain the desired hair color tone previously acquired at the salon. A purpose of this example composition is to cancel out the brassy tones that would usually appear after two weeks of hair coloring treatment. The composition uses dextrose, FD&C Blue 1 Lake, and organic and certified mineral oils, which simultaneously hydrate and restore hair with vitamins and antioxidants.

This example product may be used as often as desired without drying out hair as purple and blue shampoos usually do. It is made of natural ingredients and is perfect for in between salon visits. Recommended for highlights, balayage, and ombre, it is a color enhancer with a healthy and beautiful shine.

This example composition includes the following oils:

Avocado oil. This oil contains monosaturated fats, making it a good choice for hair treatments. It contains vitamins A, B-5 and E, antioxidants, and minerals that can help keep hair strong and healthy.

Argan oil. Argan oil comes from the argan tree scientifically known as Argania spinosa. A plant native to Morocco. Argan oil contains oleic acid, linoleic acid, palmitic acid, stearic acid, vitamin E, and squalene. Argan oil moisturizes and conditions dry hair and scalp. The powerful antioxidants and nourishing fatty acids in argan oil help keep your hair moisturized and protects against damage and damage caused by free radicals. This can result in less breakage and shedding. It also promotes shinier hair.

Jojoba oil. Jojoba oil is the liquid extracted from the seeds of the Simmondsia chinensis plant. It contains B vitamins, C and E which helps to repair the damages caused to the follicles, as well as copper and zinc, promoting thickness and regrowth and aiding in preventing hair loss. The B vitamins such as Biotin and Niacin in this oil are excellent hair growth nutrients. They also add shine and moisture to the hair. Also, because it does a good job in sealing moisture, jojoba oil can minimize frizz.

Sweet Almond oil. Almond oil contains high levels of protein, omega 9-fatty acids, oleic acid, linoleic acid, and vitamin A, B, and E. Almond oil is an emollient, meaning it can fill in gaps in your hair at a cellular level. Using almond oil on hair gives it a softer texture over time. The oleic acid and linoleic acid found in almond oil help to strengthen and repair hair.

Grape seed oil. Grape seed oil is made by pressing grapes under high amounts of pressure, releasing the seeds' oil that is high in polyunsaturated fats and certain phytonutrients. Grape seed oil is an effective natural hair moisturizer and conditioner. It locks in moisture to the hair making it easily manageable by reducing frizz. It contains vitamin E, an antioxidant that helps to build tissue, and linoleic acid, a fatty acid which promotes hair and skin growth. It can help to restore the hair strength and shine.

Lavender essential oil. Lavender essential oil strengthens the strands. This prevents hair from breakage and actually nourishes it completely. It also repairs damaged hair. When applied to scalp, lavender oil is an anti-inflammatory, anti-fungal, and promotes hair growth.

Vanilla essential oil. Vanilla essential oil is obtained by direct extraction with the ethanol from the dried pods. It contains B vitamins especially niacin, thiamin, riboflavin, vitamin B-6, and pantothenic acid. B-complex vitamins are essential for keeping your hair strong and promoting hair growth. Vanilla beans are enriched with antioxidants and antibacterial properties. Vanilla essential oil can help keep the hair shiny.

Blue powder food coloring. It contains dextrose, which is the dextrorotatory form of glucose and the predominant naturally occurring form and FD & C Blue No. 1 Aluminum Lake is an FDA approved color commonly used to dye dosage forms such as tablets and capsules. FD & C blue No. 1 is approved for use in foods, drugs, and cosmetics. FD & C blue No. 1 is also known as Brilliant blue FCF.

This example composition is made of mineral and essential oils (for cosmetic use) and Dextrose, FD & C Blue 1 Lake. Each bottle contains a mixture of:

15 ml of 100% pure and certified organic avocado oil;

15 ml of 100% pure and certified organic argan oil;

7.5 ml of 100% pure and certified organic jojoba oil;

7.5 ml of 100% pure and certified organic almond oil;

7.5 ml of 100% pure and certified organic grapeseed oil;

3 grams of powder blue food coloring;

11 lavender essential oil drops; and

11 vanilla essential oil drops.

Instructions for use: After shampoo and conditioning, apply a small amount (about 5 to 7 drops) throughout damp hair. Style as usual. It may stain hands, clothing. The use of disposable gloves is recommended. Immediately after applying to hair, the brassiness goes away even from the first application. If this composition gets in your hands, simply wash your hands with water and soap for about 20 seconds.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An oil-based hair treatment composition comprising:
a. greater than 90 weight percent of a cosmetic oil base; and
b. less than 10 weight percent of a coloring agent.

2. The composition of claim 1, wherein the composition is configured to provide strengthening and color enhancement of hair.

3. The composition of claim 1, wherein the coloring agent is configured to be dissolved or suspended within the oil base.

4. The composition of claim 1, wherein the coloring agent comprises a blue food coloring powder.

5. The composition of claim 4, wherein the coloring powder comprises about 61 weight percent dextrose and about 39 weight percent FD&C Blue 1 Lake.

6. The composition of claim 1, wherein the cosmetic oil base comprises a plurality of pure and certified organic cosmetic oils.

7. The composition of claim 1, wherein the cosmetic oil base comprises avocado oil, argan oil, jojoba oil, almond oil, grapeseed oil, or a combination thereof.

8. The composition of claim 1, additionally comprising less than 5 weight percent essential oil.

9. The composition of claim 1, additionally comprising about 11 drops of lavender oil and about 11 drops of vanilla oil per 50 grams.

10. The composition of claim 1, wherein 50 grams of the composition comprises:
a. about 15 mL of avocado oil;
b. about 15 mL of argan oil;
c. about 7.5 mL of jojoba oil;
d. about 7.5 mL of almond oil;
e. about 7.5 mL of grapeseed oil; and
f. about 3 grams of a blue food coloring agent.

11. A method of hair treatment comprising:
a. providing an oil-based hair treatment composition comprising:
i. greater than 90 weight percent of a cosmetic oil base; and
ii. less than 10 weight percent of a coloring agent; and
b. applying about 3 to 10 drops of the composition to hair and distributing the composition throughout the hair.

12. The method of claim 11, wherein the coloring agent comprises a blue coloring agent.

13. The method of claim 11, wherein the hair is damp prior to application of the composition.

14. The method of claim 11, wherein disposable gloves are worn during application of the composition.

15. The method of claim 11, additionally comprising washing hands with water and soap for about 20 seconds after application of the composition.

16. A method of preparing an oil-based hair treatment composition, the method comprising:
a. providing one or more cosmetic oils;
b. providing one or more coloring agents; and
c. mixing the one or more cosmetic oils with the one or more coloring agents to provide an oil-based hair treatment composition.

17. The method of claim 16, wherein the one or more cosmetic oils comprise avocado oil, argan oil, jojoba oil, almond oil, grapeseed oil, or a combination thereof.

18. The method of claim 16, wherein the one or more cosmetic oils comprise greater than 90 weight percent of the composition and the one or more coloring agents comprise less than 10 weight percent of the composition.

19. The method of claim 16, wherein 50 grams of the composition comprises:
a. about 15 mL of avocado oil;
b. about 15 mL of argan oil;
c. about 7.5 mL of jojoba oil;
d. about 7.5 mL of almond oil;
e. about 7.5 mL of grapeseed oil; and
f. about 3 grams of a blue food coloring agent.

20. The method of claim 16, additionally comprising providing one or more essential oils and mixing the one or more essential oils with the one or more cosmetic oils with the one or more coloring agents.
